(19) United States
(12) Patent Application Publication
(10) Pub. No.: US 2018/0119091 A1
(43) Pub. Date: May 3, 2018

(54) ORGANOTYPIC LUNG MODEL WITH FUNCTIONAL IMMUNE CELLS

(71) Applicant: Battelle Memorial Institute, Richland, WA (US)

(72) Inventors: Becky M. Hess, Richland, WA (US); Thomas J. Weber, Richland, WA (US)

(21) Appl. No.: 15/799,869

(22) Filed: Oct. 31, 2017

Related U.S. Application Data
(60) Provisional application No. 62/414,955, filed on Oct. 31, 2016.

(51) Int. Cl. C12N 5/00; C12N 5/071

(52) U.S. Cl. CPC C12N 5/0062; C12N 2500/72; C12N 5/0688; C12N 5/0018

(57) ABSTRACT

A three dimensional in vitro co-culture system is provided for determining a pathogen's interaction with immune cells differentiated from healthy tissues and grown at air-liquid interface with epithelial cells. Also provided are methods of producing the three dimensional in vitro co-culture system. The system provides a way to assess predictive pathogenicity and threat, and develop medical countermeasures.

### CROSS REFERENCE TO RELATED APPLICATION

This application claims the benefit of the earlier filing date of U.S. Patent Application No. 62/414,955, filed Oct. 31, 2016, which is incorporated herein by reference in its entirety.

### ACKNOWLEDGEMENT OF GOVERNMENT SUPPORT

This invention was made with Government support under Contract DE-AC0576RL01830 awarded by the U.S. Department of Energy. The Government has certain rights in the invention.

### FIELD

A three dimensional in vitro co-culture system for determining a pathogen's interaction with immune cells in a lung is provided, along with methods of producing the three dimensional in vitro co-culture system, and methods of using the three dimensional in vitro co-culture system to determine a pathogen's interaction with immune cells in the lungs and to screen potential therapeutic agents against lung pathogens.

### BACKGROUND

Bacillus anthracis is a non-motile, Gram-positive, spore-forming bacterium; it is the causative agent of anthrax. B. anthracis spores are metabolically dormant, robust structures that are highly resistant to harsh environmental conditions (heat, ionizing radiation, pressure, and chemicals). B. anthracis spores are one of the more likely pathogens to be used as a bioweapon because of the ease of storage and possibility for aerial dispersal. Inhalation anthrax can occur when spores are inhaled and gain access to the deep lung. Inhaled spores germinate within the host to produce metabolically active vegetative forms; these multiply and express their virulence factors, which are toxic to the host. Because B. anthracis is non-motile, it relies on intrinsic host factors for dissemination. Spores are recognized and engulfed by alveolar macrophages and transported to regional lymph nodes (draining site of the infected tissue). Within the alveolar macrophages, the spores germinate to the vegetative form, release virulence factors, kill the host cell, and escape. The vegetative bacteria begin to proliferate and overwhelm the immune function within the lymph node, enabling access to successive nodes, and ultimately the blood stream. This leads to septicemia and death of the host.

Alveolar macrophages (AM) provide innate immune protective functions in the lung, through the expression of pattern recognition receptors which recognize antigens associated with pathogens. Antigen recognition activates the macrophage and stimulates pathogen engulfment (phagocytosis), followed by direct killing within the alveolar macrophages. The macrophages also recruit other immune cell types to the site of infection by secreting immuno-modulatory signaling molecules including cytokines, chemokines, and leukotrienes. The activated macrophages then secrete additional cytokines to activate the more specialized response of the adaptive immune system.

In medicine, quantitative risk assessment models for inhaled human pathogens continue to be hampered by a lack of human in vivo data regarding the fate of the pathogen after gaining entry to the lung. In particular, with regard to B. anthracis, for the past 20 years, pathogen biologists have used a line of immortalized human macrophages (THP-1 cells) to study the interaction between this pathogen and the immortalized macrophages. However, the present inventors discovered that these immortalized macrophages do not engulf or kill spores at physiologically relevant spore doses, in contrast to data from in vivo studies, which clearly show engulfment of spores by macrophages in the lung. This finding calls into question the suitability of immortalized macrophages as a model system for evaluating host responses to B. anthracis spores, and for testing medical countermeasures.

What is needed are physiologically relevant in vitro lung tissue models that replicate systems from humans and animals that can be challenged with relevant pathogens to gather biokinetic data on pathogen fate.

### SUMMARY

The present application answers to this need by providing an in vitro system that uses only primary (e.g., healthy) tissues, as opposed to immortalized cells. In contrast to traditional undifferentiated monolayers of immortalized cells, which are continuously submerged in culture medium, the primary tissues used in the devices and methods provided herein are differentiated into mature macrophages and dendritic cells with immune function, and cultured at air-liquid interface with epithelial cells (such as lung epithelial cells).

Thus, a three dimensional in vitro co-culture system for determining a pathogen's interaction with immune cells in a lung is provided. In one example, the system includes a co-culture of mature macrophages and dendritic cells at air-liquid interface with epithelial cells.

In one example, the macrophages and dendritic cells are differentiated from peripheral blood mononuclear cells (PBMCs) (for example from a mammal, such as a human, mouse, rat or rabbit) prior to addition to the epithelial cells in the three dimensional in vitro co-culture system. Differentiated macrophages express CD14 and differentiated DCs express CD11c.

In one example, the epithelial cells are isolated from the bronchi, the trachea or the lungs of the subject (such as a mammalian subject), cultured and added to the three dimensional in vitro system. The ratio of macrophages and dendritic cells to epithelial cells can be from about 1 macrophage or dendritic cell for every 10 epithelial cells to about 1 macrophage or dendritic cell for every 1000 epithelial cells.

In one example, the three dimensional in vitro co-culture system is infected with a pathogen, such as one or more of Bacillus anthracis, Francisella tularensis, Mycobacterium tuberculosis, or Pseudomonas aeruginosa. In one example, the pathogen is Bacillus anthracis. In an additional example, the macrophages and dendritic cells express a differentiated immune cell's marker, and the three dimensional in vitro co-culture system may express mucus as a result of the infection. In yet another example, the macrophages and dendritic cells have immune activity and inactivate the pathogen or reduce infection with the pathogen.

Also provided are methods of producing a three dimensional in vitro co-culture system. The method can include isolating epithelial cells from normal bronchial, tracheal or lung tissue obtained from a subject; growing the epithelial cells in a culture medium on a permeable support at air-liquid interface; differentiating PBMCs or other immature immune cells obtained from a subject into mature macrophages that express CD14 and mature DCs that express CD11c; adding the mature macrophages and DCs to the epithelial cells at air-liquid interface; and co-culturing the mature macrophages, DCs and epithelial cells at air-liquid interface, thereby producing a three dimensional in vitro co-culture system.

In one example, the PBMCs are matured into macrophages by treatment or incubation with phorbol myristate acetate or macrophage colony-stimulating factor (MCSF). In another example, the PBMCs are matured into dendritic cells by treatment or incubation with granulocyte-macrophage colony-stimulating factor (GMCSF), in the presence of glutamine and interleukin 4 (IL4).

In one example, the ratio of macrophages and dendritic cells to epithelial cells can be from about 1 macrophage or dendritic cell for every 10 epithelial cells to about 1 macrophage or dendritic cell for every 1000 epithelial cells.

The method can further include infecting the three dimensional in vitro co-culture system with a pathogen such as one or more of Bacillus anthracis, Francisella tularensis, Mycobacterium tuberculosis, or Pseudomonas aeruginosa. In one example, the pathogen is Bacillus anthracis. The macrophages and dendritic cells may express a differentiated immune cell's marker, and the three dimensional in vitro co-culture system may express mucus as a result of the infection.

In yet another example, the macrophages and dendritic cells have immune activity and inactivate the pathogen or reduce infection with the pathogen.

Also provided are methods of determining a pathogen's interaction with immune cells in a lung. The method can include infecting the disclosed three dimensional in vitro co-culture system with a pathogen (or a pathogenic spore) and identifying molecular targets in the macrophages and dendritic cells that are necessary for pathogen spore recognition and engulfment, thereby determining the pathogen's interaction with immune cells. In one example, the pathogen is one or more of Bacillus anthracis, Francisella tularensis, Mycobacterium tuberculosis, and Pseudomonas aeruginosa.

Also provided are methods of screening a test therapeutic agent for its effectiveness against lung pathogens. The methods may include adding the test agent to the disclosed three dimensional in vitro co-culture system, heating part of the culture to 70° C. to obtain heat-resistant spores of the pathogen; calculating the total number of bacteria and the total number of heat-resistant spores; and determining the effect of the test agent on pathogen spore germination or on pathogen vegetative proliferation, or both.

The foregoing and other objects and features of the disclosure will become more apparent from the following detailed description, which proceeds with reference to the accompanying figures.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 provides a schematic comparison of an air-liquid interface culture (left panel) versus a standard cell culture (right panel). Left panel: An insert 1 containing a permeable solid support 7 (such as a Transwell® permeable solid support) is present in a cell culture vessel 9 (lid is not shown), such that the permeable solid support 7 is in contact with, but not submerged in, cell culture medium 8. The permeable solid support 7 allows cell culture medium 8 to contact epithelial cells 6 grown on the permeable solid support 7. The immune cells (such as mature macrophages and dendritic cells) 2 are seeded onto the epithelial cells 6, resulting in a system where the epithelial cells 6 and the immune cells 2 are at an air-interface 10. Pathogen spores or pathogen 4 can be added to the in vitro culture on top of the immune cells 2, and allowed to diffuse into the system, resulting in the immune cells phagocytosing pathogen or spores 5, and the production of mucus 3. Right panel: Cell culture vessel 11 (lid is not shown) containing cell culture medium 12 and adherent epithelial cells 13 (e.g., lung epithelial cells) submerged in the cell culture medium 12.

FIGS. 2A and 2B show THP-1 cells (FIG. 2A) and macrophages differentiated from human PBMCs (FIG. 2B) phagocytosing Bacillus anthracis spores and vegetative forms (shown in red). The images are confocal slices through the middle of the cells showing internalized anthrax spores or vegetative forms.

FIGS. 3A-3C provide a comparative analysis of immortalized human macrophages (THP-1 cells) versus microphages differentiated from primary healthy tissue according to the methods described herein. FIG. 3A shows that THP-1 cells associate with, but do not engulf spores. Phagocytic function was confirmed by uptake of nanoparticles (fluorescent beads). FIG. 3B shows that THP-1 cells engulf the vegetative form of Bacillus anthracis and fluorescently labeled Escherichia coli. FIG. 3C shows that primary microphages differentiated from peripheral blood mononuclear cells engulf spores and vegetative Bacillus anthracis.

FIG. 4 provides a schematic representation of the method used to produce the three dimensional in vitro co-culture system disclosed herein. Macrophages and dendritic cells are differentiated from PBMCs from a subject (such as a mammal, here a rabbit) prior to addition to the epithelial cells in the disclosed three dimensional in vitro co-culture system at air-liquid interface, and separately from the epithelial cells. The epithelial cells are isolated from the bronchi, trachea or lungs of the subject, cultured, and added into the three dimensional in vitro co-culture system. Upon infection with Bacillus anthracis, the macrophages and dendritic cells express sialic acid, and the three dimensional in vitro co-culture system expresses mucus as a result of the infection.

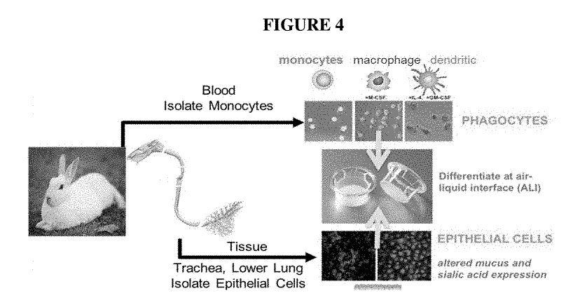

FIG. 5 provides an overview of the disclosed lung organotypic culture system that includes primary lung epithelial cells co-cultured with functional macrophages and dendritic cells at an air liquid interface (ALI). Growth media formulations were used that supported mammalian cell health without artificially accelerating bacterial growth. Primary lung epithelial cells from each species (NZW rabbit or human) were cultured on permeable solid supports (e.g., Transwell® permeable solid supports from Corning) and brought to ALI. The epithelial cells express mucus/surfactant proteins as shown with MUC5A staining (i), adopt three dimensional structures as shown in the bright field image (ii), form tight junctions as shown with CellMask Orange plasma membrane stain (ii), and are positive for sialic acid differentiation markers (iv; sialic acid is pink, DAPI stain is blue). PBMCs are differentiated into mature macrophages that express CD14 and dendritic cells that express CD11c using appropriate differentiating stimuli for each respective cell type. The differentiated macrophages and DCs engulf Bacillus anthracis vegetative bacteria (v) and spores (vi). Growth media is Dulbecco's Modified Eagle Medium containing 1% species-specific serum that was heat-inactivated.

FIG. 6 shows differences between the disclosed three dimensional in vitro co-culture system (first row) and standard two dimensional immortalized cell platform (second row). Upon infection with Bacillus anthracis, macrophages and dendritic cells express sialic acid (α-2,6 and α-2,3 sialic acid), and the disclosed three dimensional in vitro co-culture system expresses mucus as a result of the infection. These features are not observed in the standard two dimensional immortalized cell platform. These data demonstrate that the disclosed three dimensional in vitro co-culture system is a superior platform for in vitro testing pathogenesis.

FIG. 7 provides a comparison of epithelial cells alone at air-liquid interface (left panel) versus the three dimensional in vitro co-culture system comprising epithelial cells, macrophages and dendritic cells at air-liquid interface (right panel). Each of the two platforms was challenged with 170 Bacillus anthracis spores/cm². The epithelial cells alone only engulfed the vegetative form of Bacillus anthracis, and spore germination and proliferation was observed in the medium. In the three dimensional in vitro co-culture system, the macrophages and dendritic cells internalized the Bacillus anthracis spores, thus killing the pathogen.

FIGS. 8A and 8B provide a comparison of the effect of the growth media alone (first panel), epithelial cells alone grown at air-liquid interface (second panel), macrophages and dendritic cells alone (third panel), and the three dimensional in vitro co-culture system comprising epithelial cells, macrophages and dendritic cells at air-liquid interface (fourth panel) on Bacillus anthracis spore proliferation in the human (FIG. 8A) and rabbit (FIG. 8B) system. Each of the four platforms was challenged with 170 Bacillus anthracis spores/cm². The effect of the immune cells (macrophages and dendritic cells) on the pathogen was measured at three different ratios: 1:10 (1 immune cell for every 10 epithelial cells-highest ratio); 1:20 (1 immune cell for every 20 epithelial cells); and 1:40 (1 immune cell for every 410 epithelial cells-lowest ratio). The data indicate that killing of B. anthracis by the immune cells increased with increasing numbers of immune cells for both the human and the rabbit systems. These data also indicate that even at the lowest ratio (1:40) the macrophages and dendritic cells differentiated from healthy tissue retain immune function and reduce pathogen growth compared to epithelial cells alone.

### DETAILED DESCRIPTION

The disclosed three dimensional in vitro co-culture system enables macrophages and dendritic cells differentiated from primary healthy tissue to retain their immune cell function at an air liquid interface with primary epithelial cells (e.g., from lung). The macrophages and dendritic cells with immune function are differentiated from PBMCs (e.g., from a mammal, such as a human), harvested from their monoculture state, and then added to primary epithelial cells (e.g., from lung) grown at air-liquid interface. The macrophages and dendritic cells thus developed retain their immune function and inactivate lung pathogens in the system. Lung pathogens include, but are not limited to, Bacillus anthracis, Francisella tularensis, Mycobacterium tuberculosis, and Pseudomonas aeruginosa.

The three dimensional in vitro co-culture system provides for the first time a system where immune cells derived from a primary source are active against a pathogen at air-liquid interface in the presence of primary epithelial cells and physiologically relevant doses and cell numbers of the pathogen.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising an epithelial cell” means “including an epithelial cell” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entirety.

In order to facilitate review of the various embodiments of the disclosure, the following explanations of specific terms are provided:

Air-liquid interface (ALI): includes a system where the basal surface of a permeable support is in contact with a liquid culture/growth medium, whereas the apical surface of the permeable support is exposed to air. In one example, epithelial cells are seeded onto a scaffold that contains a permeable solid support, such as a Transwell® permeable solid support from Corning. The scaffold is initially supplied with culture medium to both the apical and basal compartments. Once confluence of the epithelial cells is reached, the cells are air-lifted, and the medium is supplied only to the basal chamber. Immune cells (e.g., macrophages and dendritic cells) previously differentiated from PBMCs or other immature immune cells are then added to the ALI on top of the epithelial cells. This configuration mimics the conditions found in the human airway and promotes cell differentiation.

Cell culture: refers to the maintenance of cells in an artificial, in vitro environment, or the maintenance of cells in an external, ex vivo environment (i.e., outside of an organism), and can include the cultivation of individual cells and tissues, for example, in or on a culture/growth media. Primary cells can be isolated using a single needle biopsy, a tissue biopsy, or isolated from body fluids (e.g., body-cavity fluids, or from the circulation of a subject, such as blood or fractions thereof). After isolation, the cells may be washed, treated with an enzymatic solution, concentrated, centrifuged with sufficient force to separate the cells and washed with buffer and/or saline and/or cell culture medium. The centrifuging and washing can be repeated any number of times. After a final washing, cells can then be washed with any suitable cell culture medium. Cells may or may not be counted using an electronic cell counter, such as a Coulter Counter, or they can be counted manually using a hemocytometer. Cell seeding densities may be adjusted according to certain desired culture conditions. Cells may be cultivated in a cell incubator at about 37° C. at normal atmospheric pressure. The incubator atmosphere may be humidified and may contain from about 3-10% carbon dioxide in the air. In some instances, the incubator atmosphere may contain from about 0.1-30% oxygen. Temperature, pressure and carbon dioxide and oxygen concentration may be altered as needed. Culture medium pH may be in the range of about 7.1 to about 7.6, or from about 7.1 to about 7.4, or from about 7.1 to about 7.3. Cell culture medium may be replaced every 1-2 days or more or less frequently as needed. As the cells approach confluence in the culture vessel, they may be passaged. A cell passage is a splitting or dividing of the cells, and a transferring a portion of the cells into a new culture vessel or culture environment. Cells which are adherent to the cell culture surface may require detachment, for example using enzymes such as trypsin.

Cell Culture (or Growth) Medium: may include any type of medium such as, for example, a serum-free medium; a serum-containing medium (such as a serum of the same species of the cells present in the medium); a reduced-serum medium; a protein-free medium; a chemically defined medium; a protein-free, chemically defined medium; a peptide-free, protein-free, chemically defined medium; an animal protein-free medium; a xeno-free medium. A cell culture medium typically is an aqueous-based medium and can include any of the commercially available and/or classical media such as, for example, Dulbecco's Modified Essential Medium (DMEM), Knockout-DMEM (KODMEM), Ham's F12 medium, DMEM/Ham's F12, Advanced DMEM/Ham's F12, Ham's F-10 medium, RPMI 1640, Eagle's Basal Medium (EBM), Eagle's Minimum Essential Medium (MEM), Glasgow Minimal Essential Medium (G-MEM), Medium 199, Keratinocyte-SFM (KSFM; Gibco/Thermo-Fisher), CHO cell culture media, PER.C6 media, 293 media, hybridoma media, and the like and combinations thereof. In some embodiments, a cell culture medium is a serum-containing medium. Serum may include, for example, fetal bovine serum (FBS), fetal calf serum, goat serum or human serum. Generally, serum is present at between about 1% to about 30% by volume of the medium. In some instances, serum is present at between about 0.1% to about 30% by volume of the medium. In some embodiments, a medium contains a serum replacement. In some embodiments, a cell culture medium is a serum-free medium. A serum-free medium generally does not contain any animal serum (e.g., fetal bovine serum (FBS), fetal calf serum, goat serum or human serum), but may contain certain animal-derived products such as serum albumin (e.g., purified from blood), growth factors, hormones, carrier proteins, hydrolysates, and/or attachment factors. In some embodiments, a serum-free cell culture medium comprises Keratinocyte-SFM (KSFM; Gibco/Thermo-Fisher). KSFM may include insulin, transferrin, hydrocortisone, Triiodothyronine (T3). In some embodiments, a cell culture medium is a defined serum-free medium. Defined serum-free media, sometimes referred to as chemically-defined serum-free media, generally include identified components present in known concentrations, and generally do not include undefined components such as animal organ extracts (e.g., pituitary extract) or other undefined animal-derived products (e.g., unquantified amount of serum albumin (e.g., purified from blood), growth factors, hormones, carrier proteins, hydrolysates, and/or attachment factors). Defined media may include a basal media such as, for example, DMEM, F12, or RPMI 1640, containing one or more of amino acids, vitamins, inorganic acids, inorganic salts, alkali silicates, purines, pyrimidines, polyamines, alpha-keto acids, organosulphur compounds, buffers (e.g., HEPES), antioxidants and energy sources (e.g., glucose); and may be supplemented with one or more of recombinant albumin, recombinant growth factors, chemically defined lipids, recombinant insulin and/or zinc, recombinant transferrin or iron, selenium and an antioxidant thiol (e.g., 2-mercaptoethanol or 1-thioglycerol). Recombinant albumin and/or growth factors may be derived, for example, from non-animal sources such as rice or E. coli, and in certain instances synthetic chemicals are added to defined media such as a polymer polyvinyl alcohol which can reproduce some of the functions of bovine serum albumin (BSA)/human serum albumin (HSA). In some examples, a cell culture medium is a xeno-free serum-free medium. Xeno-free generally means having no components originating from animals other than the animal from which cells being cultured originate. For example, a xeno-free culture has no components of non-human animal origin when human cells are cultured. In some embodiments, a cell culture medium is a defined xeno-free serum-free medium. Defined xeno-free serum-free media, sometimes referred to as chemically-defined xeno-free serum-free media, generally include identified components present in known concentrations, and generally do not include undefined components such as animal organ extracts (e.g., pituitary extract) or other undefined animal-derived products (e.g., serum albumin (e.g., purified from blood), growth factors, hormones, carrier proteins, hydrolysates, and/or attachment factors). Defined xeno-free serum-free media may or may not include lipids and/or recombinant proteins from animal sources (e.g., non-human sources) such as, for example, recombinant albumin, recombinant growth factors, recombinant insulin and/or recombinant transferrin. Recombinant proteins may be derived, for example, from non-animal sources such as a plant (e.g., rice) or bacterium (e.g., E. coli), and in certain instances synthetic chemicals are added to defined media (e.g., a polymer (e.g., polyvinyl alcohol)), which can reproduce some of the functions of bovine serum albumin (BSA)/human serum albumin (HSA).

Cell growth: refers to cell division or cell expansion. Stimulation of cell growth can be assessed by plotting cell populations over time.

Dendritic cell (DC): refers to an antigen-presenting cell existing in vivo, in vitro, ex vivo, or in a host or subject, or which can be derived from a hematopoietic stem cell or a monocyte. DCs and their precursors can be isolated from a variety of lymphoid organs (e.g., spleen, lymph nodes), as well as from bone marrow and peripheral blood. DCs can induce antigen specific differentiation of T cells in vitro, and are able to initiate primary T cell responses in vitro and in vivo. The term “DCs” includes differentiated dendritic cells. These cells can be characterized by expression of certain cell surface markers, such as CD11c, MHC class II, and at least low levels of CD80 and CD86. In addition, DCs can be characterized functionally by their capacity to stimulate alloresponses and mixed lymphocyte reactions (MLR).

Effective amount or therapeutically effective amount: refers to an amount of drug or therapeutic agent that is sufficient to achieve the intended effect including, but not limited to, disease treatment, disease reduction, disease prevention, reduction of spore germination, reduction of spore proliferation, and pathogen killing. The therapeutically effective amount may vary depending upon the intended application (in vitro or in vivo), or the subject and disease condition being treated, e.g., the weight and age of the subject, the severity of the disease condition, the manner of administration and the like, which can readily be determined. The term also applies to a dose that will induce a particular response in target cells, e.g., reduction of spores. The specific dose can vary depending on the particular method of administration of the therapeutic agent or drug, the dosing regimen to be followed, whether it is administered in combination with other compounds, timing of administration, the tissue to which it is administered, and the physical delivery system in which it is carried.

Epithelial cells: include squamous epithelial cells, columnar epithelial cells, adenomatous epithelial cells or transitional epithelial cells. Epithelial cells can be arranged in single layers or can be arranged in multiple layers, depending on the organ and location. Epithelial cells of the respiratory tract are non-keratinocyte (NKE) epithelial cells. NKE cells typically differentiate into functional, viable cells which function, for example, in absorption and/or secretion. The flat type I epithelial cells cover 95% of the peripheral lung surface and reside in close proximity to capillary beds and thus is the site of gas exchange. The epithelial cells of the lung provide structural integrity, are a physical barrier against environmental insults, allow gas exchange, enhance ion and fluid transport, secrete growth factors, chemo-attractants, antimicrobials, and express adhesion receptors, oxidant species, and lipid mediators for neighboring cell communication and matrix attachment. Interruption of the creation of the specialized epithelial cell types negatively impacts on lung morphogenesis. Epithelial cells may be obtained from a subject and/or a cellular source, generally referred as a primary epithelial cell population. A cellular source may include a population of embryonic stem (ES) cells, induced pluripotent stem cells (iPSCs), and the like. A primary epithelial cell population can be obtained from a subject in a variety of manners (e.g., harvested from living tissue, such as a biopsy, body fluids such as mucus, or isolated from circulation). A sample can include any specimen that is isolated or obtained from a subject or part thereof.

Immune response: includes, but is not limited to, the induction or activation of antibodies, neutrophils, monocytes, macrophages (including both M1-like macrophages and M2-like macrophages), B cells, T cells (including helper T cells, natural killer cells, and cytotoxic T cells). In one embodiment, the response is specific for a particular antigen (an “antigen-specific response”), such as a pathogenic antigen, such as Bacillus anthracis, Francisella tularensis, Mycobacterium tuberculosis, or Pseudomonas aeruginosa. In one embodiment, an immune response is a T cell response, such as a CD4+ response or a CD8+ response. A “parameter of an immune response” is any particular measurable aspect of an immune response, including, but not limited to, cytokine secretion (IL-6, IL-10, IFN-α, etc.), immunoglobulin production, dendritic cell maturation, and proliferation of a cell of the immune system. “Reducing or inhibiting an immune response” includes the use of any composition or method that results in a decrease in any of these parameters.

Inflammatory response: characterized in vivo by redness, heat, swelling and pain (i.e., inflammation) and typically involves tissue injury or destruction. An inflammatory response is usually a localized, protective response elicited by injury or destruction of tissues, which serves to destroy, dilute or wall off (sequester) both the injurious agent and the injured tissue Inflammatory responses are notably associated with the influx of leukocytes and/or leukocyte (e.g., neutrophil) chemotaxis. Inflammatory responses may result from infection with pathogenic organisms and viruses, noninfectious means such as trauma or reperfusion following myocardial infarction or stroke, immune responses to foreign antigens, and autoimmune diseases Inflammatory responses encompass conditions associated with reactions of the specific defense system as well as conditions associated with reactions of the non-specific defense system. In the lungs, inflammation is characterized by redness, swelling, heat, pain, and loss of function. It is also accompanied by vasodilation, increased vascular permeability, and inflammatory cell infiltration Inflammatory responses are to destroy and remove, as well as to wall off and confine the injurious agents. Furthermore, inflammation stimulates the immune response to promote recovery. Acute lung inflammation is dominated by neutrophils, whereas chronic reactions involve mainly macrophages and lymphocytes. As used herein, the disclosed system detects an inflammatory response in the lungs by assessing the immune response of macrophages and dendritic cells.

Bacterial Infections

When the lung is exposed to minimal bacterial loads, pathogen clearance operates through innate defenses and the event is generally subclinical. Acute infection results when higher loads of bacteria overcome the local defenses, leading to acute inflammation involving both innate and adaptive defenses. Bacterial colonization results from abnormal innate defenses, establishing an equilibrium between bacterial replication and clearance. Chronic infection occurs when an inflammatory response generated by host defense mechanisms fails to clear the bacteria, with continued tissue destruction. When inhaled in a significant load, bacteria overcome primary host defenses by releasing ciliary toxins, pneumolysin, endotoxin, and IgA proteases, thereby disrupting mucociliary clearance. Ultimately, bacteria adhere to the epithelium. In response, dendritic cells, alveolar macrophages, and epithelial cells are activated as pathogen markers are identified through toll like receptors (TLRs). Recognition of the pathogen initiates inflammation, which progresses through four phases: initiation, amplification, phagocytosis, and resolution.

Isolated: An “isolated” biological component, such as a cell (such as a PBMC, epithelial cell, macrophage, or dendritic cell), nucleic acid, protein or organelle, has been substantially separated or purified away from other biological components in the environment in which the component occurs, e.g., other cells, chromosomal and extra-chromosomal DNA and RNA, proteins and organelles. Cells, nucleic acids and proteins that have been “isolated” include cells, nucleic acids and proteins purified by standard purification methods.

Macrophage: refers to a white blood cell involved in the control of the immune response and capable of phagocytosis. Upon digestion of the pathogen by the macrophage, an antigen of the pathogen is integrated into and presented on the macrophage's cell membrane with an MHC class II molecule. Antigen presentation results in the production of antibodies. Mature or differentiated macrophages express differentiated immune cell markers, such as CD14, and are capable of functioning as immune cells in response to a stimulus.

Mucus: refers to a usually clear viscid fluid that is secreted by mucous membranes in various tissues of the body, including by the respiratory, gastrointestinal, and reproductive tracts. Mucus moistens, lubricates and protects the tissues from which it is secreted. It includes mucin macromolecules (including mucus proteins, nucleic acids and carbohydrates), which are the gel-forming constituents of mucus. Mucus proteins include, but are not limited to, respiratory mucus proteins and digestive tract mucus proteins. The viscoelastic properties of normal mucus are dependent on the concentration, molecular weight, and degree of entanglement between mucin polymers. Mucus is a primary component of sputum, and as such, the presence of excessively viscous mucus results in a sputum which is itself excessively viscous.

Pathogen: includes pathogenic microbes or bacteria causing diseases such as plague, tuberculosis and anthrax; protozoa causing diseases such as malaria, sleeping sickness and toxoplasmosis; fungi causing diseases such as ringworm, candidiasis or histoplasmosis; and bacteria causing diseases such as sepsis. In one example, the pathogen is one found in the lung. Exemplary pathogens include, but are not limited to, Bacillus anthracis, Francisella tularensis, Mycobacterium tuberculosis, and Pseudomonas aeruginosa.

Primary cells: refers to cells that are cultured directly from an animal or person. In contrast to immortalized cell lines that have acquired the ability to proliferate indefinitely either through random mutation or deliberate modification, most primary cell cultures have limited lifespan. After a certain number of population doublings, primary cells undergo the process of senescence and stop dividing, while generally retaining viability. Primary cells may be derived from intestinal epithelial cells, or epithelial cells of the respiratory tract, endothelial cells, kidney cells, at synaptic junctions and on monocytes, macrophages, dendritic cells (DC) and granulocytes, and activated T lymphocytes.

Sample: includes any sample in which it is desirable to test for the presence of bacteria. Thus, the sample is generally a sample suspected of containing, or in some circumstances known to contain, a microorganism. A sample may comprise, consist essentially of or consist of a clinical sample, or an in vitro assay system for example. Exemplary samples include blood samples (or a fraction thereof, such as plasma or serum), sputum, saliva, biopsy material, and the like.

Standard two dimensional immortalized cell platform: a submerged in vitro system for the in vitro study of monocyte and macrophage functions and responses to pathogen infections, which includes murine derived RAW 264.7 cells or the immortalized human leukemia cell line THP-1.

Subject: refers to a human or animal. Animals include vertebrates, such as a primate, rodent, or domestic animal. Primates include chimpanzees and monkeys. Rodents include, but are not limited to, mice, rats, rabbits and hamsters. Domestic animals include, but are not limited to, cats, dogs, cows, horses, pigs, deer, birds and fish. In some embodiments, the subject is a mammal. The mammal can be a human, or a non-human primate, such as a mouse, rat, rabbit, dog, cat, horse, or cow. In some embodiments, the subject is a patient, a subject suspected of having an infection, or a subject in need of diagnosis for infection, such as a lung infection.

Therapeutic effect: refers to a therapeutic and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

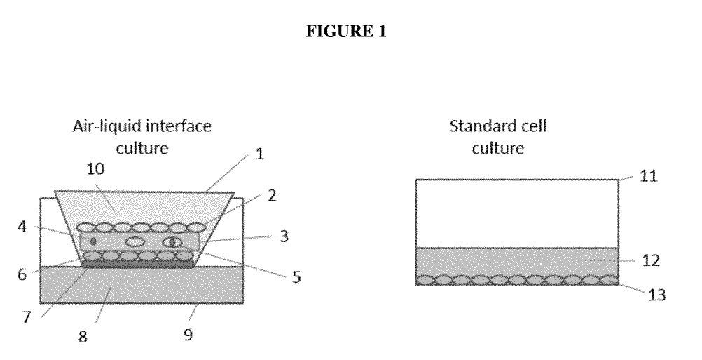

Three dimensional co-culture system: refers to a non-submerged in vitro system that includes a co-culture of mature macrophages and dendritic cells at air-liquid interface with epithelial cells, as described herein and in FIG. 1.

Treat, treatment, or treating: refers to therapeutic treatments, which may reverse, alleviate, or ameliorate the symptoms of a particular disease or disorder; inhibit, slow down or stop the progression or severity of a condition associated with a disease or disorder; or diminish, stop, slow down, or hamper spore proliferation and/or vegetative growth of a pathogen.

### Three-Dimensional System

A three dimensional system for in vitro testing is provided. The system provides a practical and reliable alternative to in-vivo testing of pathogenesis. The system utilizes primary epithelial cells, such as bronchial cells, tracheal cells and/or lung cells obtained from a subject, such as a mammalian subject; and immune cells, such as mature macrophages and dendritic cells (e.g., derived from primary PBMCs). The epithelial cells, macrophages, and dendritic cells are cultured together (e.g., co-cultured) at an air-liquid interface, and remain in contact with one another. The mature macrophages and dendritic cells thus differentiated and co-cultured retain their immune function and kill pathogens. In some examples, the epithelial cells, mature macrophages and dendritic cells used in the system are from the same species (e.g., are all human cells, are all mouse cells, or are all rabbit cells). In some examples, the epithelial cells, mature macrophages and dendritic cells used in the system are from different species (e.g., human epithelial cells, rabbit macrophages and rabbit dendritic cells; rabbit epithelial cells, human macrophages and rabbit dendritic cells).

Referring to FIG. 1, the system utilizes or includes a cell culture vessel 9 (such as a tissue culture plate, such as a multi-well plate), and an insert 1 containing a permeable solid support 7, such as a membrane (examples include a Transwell® permeable solid support from Corning). In some examples, the permeable solid support 7 is coated with collagen to promote adherence of epithelial cells. For example, the permeable solid support 7 can be treated with collagen (such as human or bovine collagen) that results in a biologically stabilized collagen matrix covering fibrils of the permeable solid support 7. In some examples, permeable solid support 7 includes a polycarbonate or polyester membrane. In some examples, permeable solid support 7 includes a polytetrafluoroethylene (PTFE) membrane. In some examples, the cell culture vessel 9 and/or the non-permeable solid support part of insert 1 are composed of polystyrene.

The system also includes primary epithelial cells (FIG. 1, see 6), such as those isolated or obtained from normal (e.g., non-diseased, for example that is free from pathogenic infection or cancer or other disorder) bronchial, tracheal or lung tissue, squamous epithelial cells, columnar epithelial cells, adenomatous epithelial cells or transitional epithelial cells, keratinocyte epithelial cells (KE) and non-keratinocyte (NKE) epithelial cells. The isolated primary epithelial cells present on the permeable solid support 7 are at about 100% confluence and are differentiated before being brought in contact with the immune cells 2 (e.g., mature macrophages and DCs.) In some examples, the culture medium 8 is Dulbecco's Modified Eagle Medium (DMEM) that includes at least 1% serum and 2 mM L-glutamine.

The system also includes immune cells (FIG. 1, see 2), such as mature macrophages (e.g., that express CD14) and mature DCs e.g., that express CD11c). In some examples, the immune cells 2 are obtained from immature immune cells (e.g., PBMCs) that are differentiated into mature macrophages that express CD14 and into mature DCs that express CD11c. Subsequently the immune cells 2 (e.g., mature macrophages and DCs) are deposited on the epithelial cells 6 on the apical side of the air-liquid interface co-culture system, for example applied to confluent, differentiated epithelial cells 6, present on a permeable substrate 7 (in contact with culture media 8 on the basal side of the epithelial cells 6). The apical side of the system corresponds to the lung surface in contact with air under normal physiological conditions. The basal side of the system corresponds to the lung surface in contact with the body of the subject under normal physiological conditions. In some examples, the ratio of macrophages and dendritic cells to epithelial cells is from about 1 macrophage or dendritic cell for every 10 epithelial cells to about 1 macrophage or dendritic cell for every 40 epithelial cells, or for every 50 epithelial cells, or for every 100 epithelial cells, or for every 500 epithelial cells, or for every 1000 epithelial cells. The resulting system allows the production of mucus 3 from epithelial cells 6 following introduction of a pathogen or spore 4, and provide a physiologically relevant system.

During use, the three dimensional in vitro co-culture system can be infected with a pathogen, such as one that can infect a lung, such as one or more of Bacillus anthracis, Francisella tularensis, Mycobacterium tuberculosis, or Pseudomonas aeruginosa, for 1 to 10 hours. In one example, the pathogen is Bacillus anthracis. In one example, spores of the pathogen are used, such as a dose range between 170 spores/cm² to 1700 spores/cm². The spores or pathogen can be provided in a liquid suspension, which is added directly to the apical side of the system containing the immune cells 2. It is shown herein that the mature immune cells 2 engulf the introduced pathogen or spore 4 (for example, Bacillus anthracis Sterne) (shown as immune cell with phagocytosed pathogen or spore 5) as would be expected in vivo. Upon infection of the co-culture system with a pathogen or spore 4, the mature macrophages and DCs express sialic acid, a marker normally expressed by differentiated immune cells, and the three dimensional in vitro co-culture system expresses mucus 3 as a result of the infection.

In contrast, prior systems (right panel of FIG. 1) utilize a cell culture vessel 11 containing cell culture medium 12 and adherent lung epithelial cells 13 submerged in the cell culture medium 12. In this prior system, there is no air interface (10 in left panel of FIG. 1). This makes addition of spores and immune cells problematic as they remain in suspension. As a result, it can be difficult for the immune cells 13 to come into contact with pathogens/spores and phagocytose them.

### Method of Making a Three-Dimensional System

Methods of producing the disclosed three dimensional in vitro co-culture system is also provided. As shown in to FIG. 1, the system includes epithelial cells 6 present on a permeable solid support 7. Thus, the method of producing the system can include isolating or obtaining primary epithelial cells from a subject (e.g., a mammal). For example, normal (e.g., non-diseased, for example that is free from pathogenic infection or cancer or other disorder) epithelial cells can be obtained, for example from the lung (e.g., from normal bronchial, tracheal or lung tissue). The resulting primary epithelial cells are then applied to the permeable solid support 7 (e.g., Transwell® permeable support, Corning), and allowed to grow to 100% confluence in the presence of a culture media 8 (such as Dulbecco's Modified Eagle Medium (DMEM) that includes at least 1% serum and 2 mM L-glutamine, or B-ALI™ growth media). At this stage, epithelial cells 6 are submerged in the culture media 8, such that the basal and apical sides of the epithelial cells 6 are in contact with the culture media 8. After reaching confluence, culture media 8 is removed (e.g., aspirated) from the apical and basal side of the permeable solid support 7, and freshly prepared differentiation media is added to the basal side of the permeable solid support 7, such that the epithelial cells 6 are at the air-liquid interface 10 (basal side in contact with culture media 8, and apical side in contact with the air 10). After allowing the epithelial cells 6 to grow to confluence on the permeable solid support 7, the epithelial cells 6, now at the ALI 10, are incubated under conditions that allow them to differentiate. For example, the basal side of the epithelial cells 6 can be exposed to culture media 8 that allows differentiation (e.g., the differentiation media at the basal side of the permeable solid support is replaced once every day for at least 12 days prior to challenge with pathogen spores), while the apical side of the permeable solid support 7 is kept dry (e.g., has no media). This allows mature immune cells 2 to be added to the confluent epithelial cells 6.

The method also includes differentiating immature immune cells, such as PBMCs, into mature immune cells 2, such as mature macrophages that express CD14, and mature DCs that express CD11c. Thus, in some examples, the method includes obtaining or isolating immature immune cells (e.g., PBMCs) from a mammal (such as a human or veterinary subject). In some examples, PBMCs are obtained from the blood of a subject. In some examples, immature immune cells (e.g., PBMCs) from primary tissue are differentiated into mature macrophages in RPMI-1640 medium containing GM-CSF. In some examples, immature immune cells (e.g., PBMCs) from primary tissue are differentiated into mature dendritic cells in RPMI-1640 medium containing GM-CSF, IL-4 and PGE2.

In some examples, differentiation into mature macrophages is achieved by treating or incubating the immature immune cells (e.g., PBMCs) with effective amounts of phorbol myristate acetate, such as from about 1.0×10⁻⁷ to about 1.6×10⁻⁷ M of phorbol myristate acetate, or effective amounts of macrophage colony-stimulating factor, such as at a concentration of at least 10 ng/ml, or at least 20 ng/ml, such as 10 to 50 ng/ml. In some examples, differentiation into mature macrophages is achieved by treating or incubating the immature immune cells (e.g., PBMCs) with GM-CSF at a concentration of at least 1 ng/ml, at least 10 ng/ml, or at least 20 ng/ml, such as 1 to 100 ng/ml, 1 to 50 ng/ml, 10 to 50 ng/ml, such as 20 ng/ml or 25 ng/ml. In some examples, the immature immune cells (e.g., PBMCs) treated with a differentiation factor are subsequently tested for expression of CD14 (e.g., using an antibody specific for CD14), for example using ELISA, flow cytometry, or microscopy, for example to confirm the generation of mature macrophages.

Thus, the immune cells 2 are differentiated separately from epithelial cells 6.

In some examples, differentiation into DCs is achieved by treating or incubating the immature immune cells with an effective amount of GM-CSF, prostaglandin E2 (PGE2), and interleukin 4. In some examples, differentiation into DCs is achieved by treating or incubating the immature immune cells (e.g., PBMCs) with GM-CSF at a concentration of at least 1 ng/ml, at least 10 ng/ml, or at least 20 ng/ml, such as 1 to 100 ng/ml, 1 to 50 ng/ml, 10 to 50 ng/ml, such as 20 ng/ml or 25 ng/ml, or 50 ng/ml; IL4 at a concentration of 1 ng/ml, at least 10 ng/ml, or at least 20 ng/ml, such as 1 to 250 ng/ml, 1 to 200 ng/ml, 10 to 200 ng/ml, such as 100 ng/ml or 200 ng/ml, or 150 ng/ml; and prostaglandin E2 (PGE2) at a concentration of about 1 μg/ml, at least 10 μg/ml, or at least 20 μg/ml, such as from about 1 μg/ml to about 100 μg/ml. In some examples, the immature immune cells (e.g., PBMCs) treated with a differentiation factor are subsequently tested for expression of CD11c (e.g., using an antibody specific for CD11c), for example using ELISA, flow cytometry, or microscopy, for example to confirm the generation of DCs.

Subsequently, the mature macrophages and DCs (immune cells 2 in FIG. 1) obtained from the immature immune cells are deposited or introduced to the apical side of the air-liquid interface co-culture system containing the confluent epithelial cells 6. This results in epithelial cells and immune cells being present at the air-liquid interface 10. The apical side of the immune cells 2 corresponds to the lung surface in contact with air 10 under normal physiological conditions. The basal side of the immune cells 2 is in contact with the epithelial cells. In some examples, the mature immune cells are collected and counted. In some examples, the ratio of mature macrophages and dendritic cells to epithelial cells is from about 1 mature macrophage or dendritic cell for every 10 epithelial cells to about 1 mature macrophage or dendritic cell for every 40 epithelial cells, or for every 50 epithelial cells, or for every 100 epithelial cells, or for every 500 epithelial cells, or for every 1000 epithelial cells.

The method can then include co-culturing the mature macrophages, DCs and epithelial cells at the air-liquid interface, thereby producing a three dimensional in vitro co-culture system.

### Method of Using the Three-Dimensional System

The three dimensional in vitro co-culture system can be used to determining a pathogen's interaction with immune cells. For example, the method can include infecting the air-liquid interface co-culture system with a pathogen (or spore), such as one that can infect a lung, such as one or more of Bacillus anthracis, Francisella tularensis, Mycobacterium tuberculosis, or Pseudomonas aeruginosa, for example for 1 to 24 hours. In some examples, the pathogen or pathogenic spores in liquid suspension are added directly to the apical side containing the immune cells. In one example, the pathogen is Bacillus anthracis, and the spores are added directly to the apical side containing the immune cells in a dose of 170 spores/cm² to 1700 spores/cm². In some examples, upon infection of the co-culture system with the pathogen, the mature macrophages and DCs express sialic acid, and the three dimensional in vitro co-culture system expresses mucus as a result of the infection. In some examples, fluorescent confocal microscopy can be used to determine spore internalization by the immune cells.

The method can further include identifying molecular targets, such as additional cell markers that are expressed on the surface of the macrophages and dendritic cells that are necessary for pathogen spore recognition and engulfment, thereby determining the pathogen's interaction with immune cells. In some examples, surface proteins can be identified using biotinylation techniques and immunofluorescence. The biotinylated surficial proteins can be separated from the remaining proteins and quantitative assays can be used to determine overall levels of receptor expression. Pro-inflammatory cytokine secretion profiles can be measured using enzyme-linked immunoassorbent assays (ELISA). In some examples, cell markers can be identified by flow cytometry. For example, a high-throughput flow cytometer can be used to screen surface antibodies on differentiated macrophages by staining the differentiated macrophages with antigen presenting cells (APC)-conjugated monoclonal antibodies specific for CD14, and determining the presence of additional cell markers on the surface of the macrophages. In another example, a high-throughput flow cytometer can be used to screen surface antibodies on differentiated dendritic cells by staining the differentiated DCs with APC)-conjugated monoclonal antibodies specific for CD11c, and determining the presence of additional cell markers on the surface of the DCs.

Also provided are methods of screening a test agent for its effect against a lung pathogen. Exemplary test agents includes proteins, small molecules, antibodies, nucleic acid molecules, and the like. Additional test agents can be identified by determining whether a test agent is recognized by the cell markers expressed on the macrophage's or dendritic cell's surface. Methods of screening a test agent can include adding one or more test agents to the disclosed three dimensional in vitro co-culture system (for example contacting the test agent with the immune cells 2). In some examples, multiple doses of a test agent, multiple test agents, or combinations thereof, are tested simultaneously or contemporaneously. The method then includes determining the effect of the test agent on pathogen spore germination or bacteria proliferation, for example by measuring spore internalization (e.g., using fluorescent confocal microscopy). Potential medical countermeasures and toxins are screened for their impact on Bacillus anthracis spore germination and proliferation as possible therapies against inhaled anthrax. In some examples, a count of the total number of bacteria is obtained in part of the sample, and the remaining sample is heated (e.g., at a temperature of at least 65° C., or at least 70° C., such as about 70° C.) to determine the number of spores in the sample. This procedure allows for the distinction between bacteria and spores. In some examples, the total number of vegetative cells is calculated by subtracting the total number of bacteria from the total number of spores that survived the heating treatment to obtain the total number of spores that germinated or proliferated. In some examples, selected molecular targets on the immune cells are inhibited using molecular inhibitors, and the effect of the inhibitor on pathogen recognition and engulfment by the immune cells is determined by fluorescent confocal microscopy. Test agents that reduce pathogen spore germination or bacteria proliferation or growth, for example by at least 10%, at least 20%, at least 30%, at least 40%, at least 50%, at least 60%, at least 70%, at least 80%, at least 90%, at least 95%, at least 99%, or 100%, as compared to no test agent, are candidate agents for treating the pathogen tested. In some examples such agents are selected for efficacy in vivo against the pathogen.

Thus, the three dimensional in vitro co-culture system provides a host-pathogen response characterization which is physiologically relevant. Furthermore, the system provides a reliable platform for screening potential therapeutics in the presence of pathogens and finding a treatment for inhalation diseases and disorders.

The disclosure is further illustrated by the following examples which should not be construed as limiting. The examples are illustrative only, and are not intended to limit, in any manner, any of the aspects described herein. Further, various changes and modifications to the disclosed embodiments, which will be apparent to those of skill in the art, can be made without departing from the spirit and scope of the present application. The following examples do not in any way limit the invention.

### EXAMPLES

### Example 1

### Construction of the Three Dimensional in vitro Co-Culture System.

### A. Seeding of Primary Epithelial Cells

Permeable supports (e.g., Transwell® permeable support, Corning)) were coated with a 30 μg/mL collagen solution in 20 mM acetic acid (9 μg of collagen/cm²) and incubated at room temperature for 1 hour. The collagen solution was gently aspirated and each well was rinsed with 150 μL of PBS. The PBS rinse was removed and discarded. Epithelial cells were harvested from T-75 flasks with 0.25% Trypsin-EDTA and the resulting cell solution was treated with Trypsin-Neutralizing Solution. The cells were pelleted by centrifuging at 300×g for five minutes and suspended in B-ALI™ medium (Lonza). The cells were counted and the concentration of viable cells was determined. The cell solution's volume was adjusted to 50,000 cells in a 100 μl volume of growth media, and 50,000 cells were seeded on the apical side of a Transwell® permeable support (Corning). 500 μl of B-ALI growth media were added to the basal side of the chamber and incubated overnight. The next day the media was removed from the apical and basal side of the Transwell® permeable support and replaced with fresh growth media (100 μl on the aplical side, 500 μl on the basal side).

### B. Differentiation at Air-Liquid Interface (ALI)

The cells were monitored every day for morphology and confluency. The cells were grown for three days after 100% confluency was reached, then airlifted to maintain the cultures at ALI. For the airlifting, the medium was removed from the apical and basal side of the Transwell® permeable support, and 500 μl of freshly prepared B-ALI™ supplemented differentiation medium (Lonza) was added to the basal side of the Transwell® permeable support, while the apical side of the permeable support was kept dry. The differentiation media was replaced every other day by aspirating the media on the basal side of the chamber and replacing it with 500 μl of freshly prepared differentiation media for at least 12 days prior to conducting a spore challenge assay.

C. Preparation of Mature Immune Cells from PBMCs

PBMCs were added into a 50 ml Falcon tube containing 28 ml of complete macrophage medium (RPMI-1640) with serum and without GM-CSF media. The tube was inverted 2-3 times to generate a homogenous cell suspension and let sit at room temperature for 10 min. The cell suspension was centrifuged at 800×g for 3.45 min to pellet the PBMCs. The medium was gently aspirated, leaving about 100 μl of supernatant atop the pellet, and the cells were suspended in the remaining solution by inverting the tube several times. 30 ml of fresh medium was then added and pipetted up and down to obtain a homogenous cell solution. 40 μl of granulate-macrophage colony-stimulating factor (GM-CSF) was added to the 30 ml cell solution to a final concentration of 25 ng/ml. 15 ml of the PBMC cell solution were transferred to a first T-75 flask with a membrane cap for macrophage differentiation, and the remaining 15 ml of the cell solution were transferred to a second T-75 for dendritic cell differentiation.

a. Macrophage Differentiation

The first T-75 flask was capped with a membrane cap and the cells were incubated at 37° C., 5% CO², 95% relative humidity (RH) for 48 hours for acclimation. Subsequently, approximately 14 ml of the medium were aspirated from the T-75 flask and transferred to a 15 ml conical tube. The solution was centrifuged at 800×g for 3.45 minutes, and the supernatant gently aspirated to leave about 100 μl of supernatant atop the pellet. The bottom of the tube was flicked by hand several times to suspend the cells in the remaining solution in the tube, 14 m of complete macrophage medium without GM-CSF were added to the cell pellet and the cells were gently suspended by pipetting. The cell solution was transferred back to the original T-75 flask, and 20 μl of GM-CSF were added to the cells in the T-75 flask. The cells were incubated at 37° C., 5% CO², 95% RH for 48 hours to reach differentiation prior to addition to the primary epithelial cells. b. Dendritic Cell Differentiation

20 μl of IL-4 were added to the cells in the second T-75 flask and the cells were incubated at 37° C., 5% CO², 95% RH for 48 hours for acclimation. Approximately 14 ml of the medium were aspirated from the T-75 flask and transferred to a 15 ml conical tube. The solution was centrifuged at 800×g for 3.45 minutes, and the supernatant was gently aspirated to leave about 100 μl of the supernatant atop the pellet. The bottom of the tube was flicked by hand several times to suspend the cells in the remaining solution in the tube. 14 ml of complete macrophage medium without GM-CSF were added to the cell pellet and the cells were gently suspended by pipetting and the solution was transferred back to the original T-75 flask. 20 μl of GM-CSF and 20 μL of IL-4 were added to the cells in the T-75 flask, and the cells were incubated at 37° C., 5% CO², 95% RH for 48 hours. These steps were repeated several times before adding 10 μl of a 2 mg/ml prostaglandin E2 (PGE2) solution to the cells in the T-75 flask. The cells were incubated at 37° C., 5% CO², 95% RH for 24 hours, and then added to the primary epithelial cells.

### Example 3

### Spore Challenge Assay

Using pathogen challenge assays, it was empirically determined that neither the epithelial cells alone nor assay media contributed to pathogen spore or vegetative cell inactivation over the course of 24 hours following infection (data not shown). A dose range between 170 spores/cm² and 1700 spores/cm² was also determined for the rabbit and human in vitro system. Reproducibility was determined in the human in vitro system by testing human response in different donors, with each donor tested with three biological replicates of the pathogen at each time point. Results of a t-test showed no significant difference in lag time or maximum pathogen growth rate between donors, indicating a reproducible and reliable biological response using the novel in vitro system.

### Example 4

### Testing Ratios of Immune Cells: Epithelial Cells

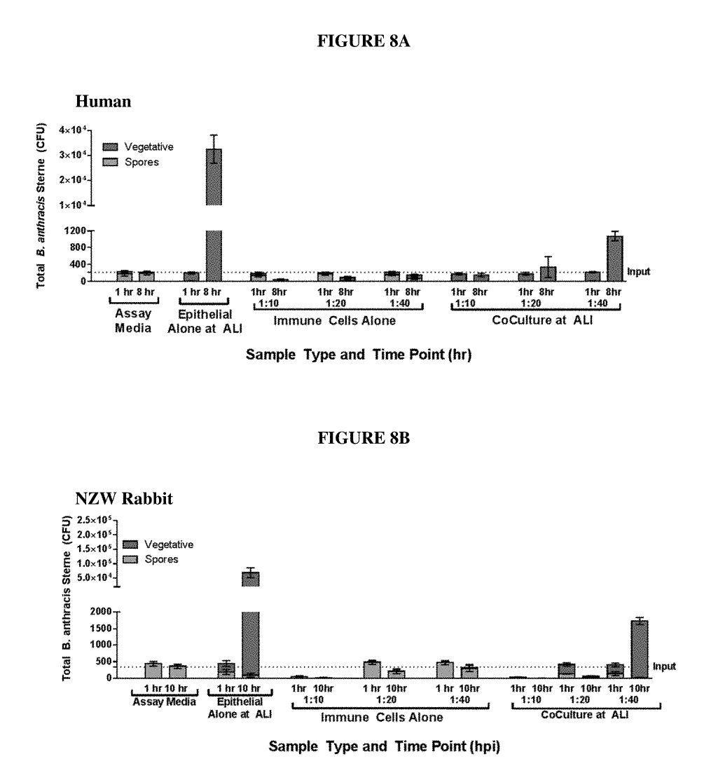

Spore challenge assays were conducted at ratios of 1 immune cell for every 10 epithelial cells, 1 immune cell for every 20 epithelial cells, and 1 immune cell for every 40 epithelial cells. The killing of B. anthracis by immune cells increased with increasing numbers of immune cells for both the NZW rabbit and human experimental lung tissue in vitro systems (FIG. 8). Based on these data, a ratio of 1 immune cell for every 40 epithelial cells was determined to be physiologically relevant in vivo.

### Example 5

### Comparison of the Three Dimensional in vitro Co-Culture System to Current in vitro Model Systems.

The immune cells most commonly used to study anthrax in vitro are the murine (mouse) derived RAW 264.7 cells or the immortalized human leukemia cell line THP-1. The RAW 264.7 cells are a poor model for the human system for two reasons: (1) infected mice do not have the same clinical presentation as humans, and (2) the Sterne strain of anthrax is lethal in mice, but is not lethal in humans. The THP-1 human cell line has been widely used to study immune response, and has been widely accepted to “behave like native monocyte-derived macrophages”.

The THP-1 cells were observed to engulf spores at extremely high doses (up to 107 spores). However, THP-1 cells were not observed to kill spores at any dose, and engulfment activity was not observed at physiologically relevant spore doses (100 to 2,000 spores).

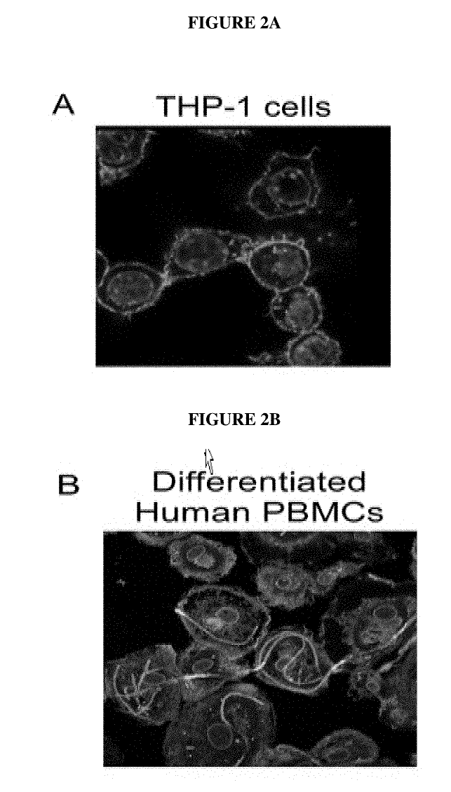

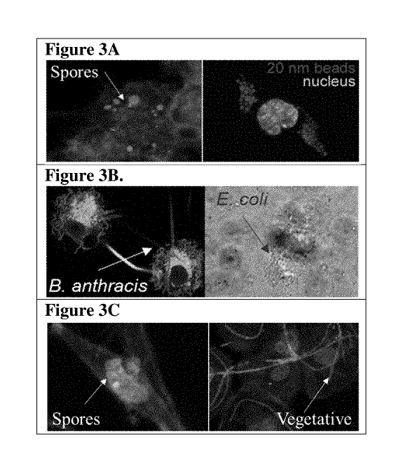

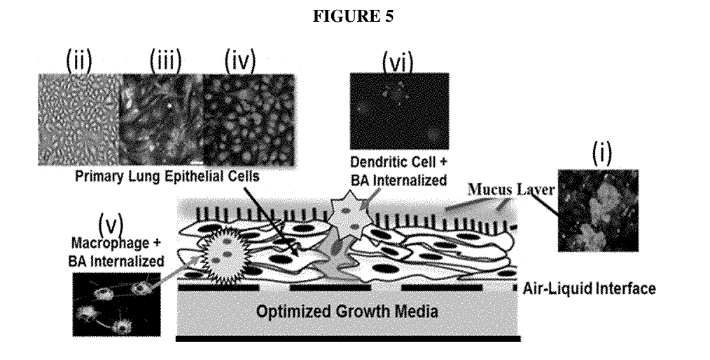

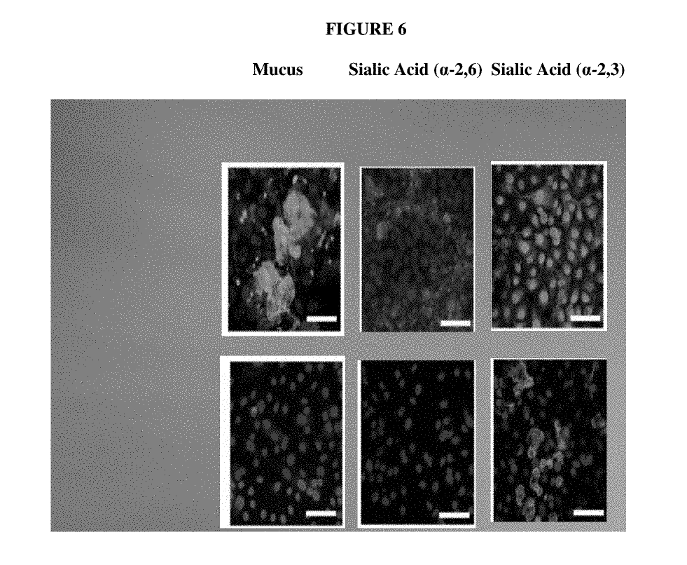

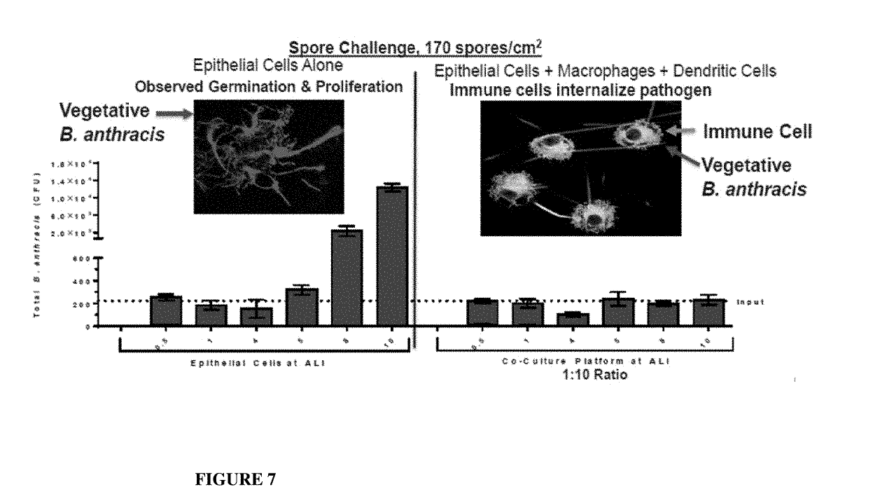

To develop a model macrophage system that could be used with in vitro studies at physiologically relevant spore doses, a maturation treatment that mimics in vivo processes, causing freshly isolated immature immune cells (e.g., PBMCs) to mature into macrophages, was developed. Epithelial cells isolated from the bronchi, trachea or lungs, and immune cells, (macrophages and dendritic cells) were cultured together at an air-liquid interface (FIG. 1, left image). Macrophages differentiated from PBMCs that were obtained from healthy rabbits and healthy humans were capable of engulfing and killing anthrax spores (FIG. 2B). Macrophages and dendritic cells co-cultured with epithelial cells at air-liquid interface internalized Bacillus anthracis spores, expressed sialic acid (α-2,6 and α-2,3 sialic acid), a marker normally expressed by differentiated immune cells, and inactivated the pathogen. Furthermore, the in vitro co-culture system expressed mucus as a result of the infection (FIGS. 5 and 6). In spore challenge assays with 170 Bacillus anthracis spores/cm², the epithelial cells alone only engulfed the vegetative form of Bacillus anthracis, and spore germination and proliferation was observed in the medium (FIG. 7). In the three dimensional in vitro co-culture system, the macrophages and dendritic cells internalized the Bacillus anthracis spores, thus killing the pathogen (FIG. 7). In fact, primary macrophages killed 90% of the spores within 10 hours post infection whereas THP-1 macrophages did not kill any of the spores (FIGS. 3C, 7 and 8). However, spores were associated with the surface of the THP-1 cells, indicating that the spores were likely bound to a THP-1 receptor, but were not being engulfed (FIG. 3A). Phagocytic capability was confirmed using nanoparticles as a positive control. The phagocytic function of the THP-1-derived macrophages was further tested by exposing the microphages to Escherichia coli and the vegetative (metabolically active) form of Bacillus anthracis Sterne. The data confirmed that the THP-1 cells actively engulfed both of these bacteria (FIG. 3B). These experiments confirmed that the THP-1 cells have phagocytic function, but fail to engulf Bacillus anthracis Sterne spores and Escherichia coli spores. These data indicate that THP-1 cells are not an appropriate model for studying spore killing by macrophages.

In contrast to the standard two dimensional immortalized THP-1 cell platform commonly used (see FIG. 1, right panel), the data herein show that the macrophages and dendritic cells differentiated from healthy tissue and cultured at air-liquid interface with epithelial cells not only engulfed, but also killed Bacillus anthracis Sterne spores. The effect of the immune cells (macrophages and dendritic cells) on the pathogen was measured at three different ratios: 1:10 (1 immune cell for every 10 epithelial cells-highest ratio); 1:20 (1 immune cell for every 20 epithelial cells); and 1:40 (1 immune cell for every 410 epithelial cells-lowest ratio) (FIG. 6). The data obtained indicate that even at the lowest ratio (1:40) the macrophages and dendritic cells differentiated from healthy tissue retain immune function and reduce pathogen growth compared to epithelial cells alone.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

What is claimed is:

1. A three dimensional in vitro co-culture system for determining a pathogen's interaction with immune cells in a lung, comprising:
an in vitro co-culture comprising mature macrophages and dendritic cells at an air-liquid interface with epithelial cells.

2. The three dimensional in vitro co-culture system of claim 1, wherein the mature macrophages and dendritic cells are differentiated from mammalian peripheral blood mononuclear cells (PBMCs) prior to addition to the three dimensional in vitro co-culture system and separately from the epithelial cells.

3. The three dimensional in vitro co-culture system of claim 2, wherein the epithelial cells are isolated from bronchi, trachea or lungs of a subject.

4. The three dimensional in vitro co-culture system of claim 3, wherein the macrophages, dendritic cells, and epithelial cells are present at a ratio from about 1 macrophage or dendritic cell for every 10 epithelial cells to about 1 macrophage or dendritic cell for every 1000 epithelial cells.

5. The three dimensional in vitro co-culture system of claim 4, wherein the three dimensional in vitro co-culture system is infected with a pathogen.

6. The three dimensional in vitro co-culture system of claim 5, wherein the pathogen is Bacillus anthracis, Francisella tularensis, Mycobacterium tuberculosis, or Pseudomonas aeruginosa.

7. The three dimensional in vitro co-culture system of claim 6, wherein the pathogen is Bacillus anthracis.

8. The three dimensional in vitro co-culture system of claim 7, wherein the macrophages and dendritic cells express a differentiated immune cell marker, and wherein the three dimensional in vitro co-culture system expresses mucus.

9. The three dimensional in vitro co-culture system of claim 8, wherein the macrophages and dendritic cells inactivate the pathogen or reduce infection with the pathogen.

10. A method of producing the three dimensional in vitro co-culture system of claim 1, comprising:
(a) isolating epithelial cells from normal bronchial, tracheal or lung tissue obtained from a subject;
(b) growing the epithelial cells in a culture medium on a membrane at air-liquid interface;
(c) differentiating PBMCs obtained from a subject into mature macrophages that express CD14 and mature DCs that express CD11c;
(d) adding the mature macrophages and DCs to the the epithelial cells at air-liquid interface; and
(e) co-culturing the mature macrophages, DCs and epithelial cells at air-liquid interface, thereby producing a three dimensional in vitro co-culture system.

11. The method of claim 10, wherein the subject is a mammal.

12. The method of claim 11, wherein the macrophages and dendritic cells are added at a ratio of about 1 macrophage or dendritic cell for every 10 epithelial cells to about 1 macrophage or dendritic cell for every 40 epithelial cells, or for every 50 epithelial cells, or for every 100 epithelial cells, or for every 500 epithelial cells, or for every 1000 epithelial cells.

13. The method of claim 12, further comprising infecting the three dimensional in vitro co-culture system with a pathogen.

14. The method of claim 13, wherein the pathogen is Bacillus anthracis, Francisella tularensis, Mycobacterium tuberculosis, or Pseudomonas aeruginosa.

15. The method of claim 13, wherein the pathogen is added to the air-liquid interface co-culture system in a dose range between 170 spores/cm² to 1700 spores/cm².

16. The method of claim 15, wherein the macrophages and dendritic cells express a differentiated immune cell's marker and wherein the three dimensional in vitro co-culture system expresses mucus.

17. The method of claim 13, wherein the macrophages and dendritic cells inactivate the pathogen or reduce infection with the pathogen.

18. A method of determining a pathogen's interaction with lung immune cells, comprising:
infecting the three dimensional in vitro co-culture system of claim 1 with a pathogen, and
identifying molecular targets in the macrophages and dendritic cells that are necessary for pathogen spore recognition and engulfment, thereby determining the pathogen's interaction with the lung immune cells.

19. The method of claim 17, wherein the pathogen is Bacillus anthracis, Francisella tularensis, Mycobacterium tuberculosis, or Pseudomonas aeruginosa.

20. A method of screening a test agent for its effect against a lung pathogen, comprising:
adding the test agent to the three dimensional in vitro co-culture system of claim 6;
heating part of the culture at about 70° C. to obtain heat-resistant spores of the pathogen; and
determining the effect of the test agent on pathogen spore germination or vegetative bacteria proliferation.
